(19) United States
(12) Patent Application Publication
(10) Pub. No.: US 2024/0164751 A1
(43) Pub. Date: May 23, 2024

(54) FLEXIBLE ELECTRONIC CIRCUIT FOR ULTRASOUND CATHETERS

(71) Applicant: St. Jude Medical, Cardiology Division, Inc., St. Paul, MN (US)

(72) Inventors: Fermin Lupotti, Lake Forest, CA (US); Arthur G. Blanck, Ramona, CA (US)

(21) Appl. No.: 18/279,781

(22) PCT Filed: Feb. 15, 2022
(86) PCT No.: PCT/US2022/016402

§ 371 Date: Aug. 31, 2023

Related U.S. Application Data
(60) Provisional application No. 63/157,232, filed on Mar. 5, 2021.

(51) Int. Cl. A61B 8/12; A61B 8/00; A61B 8/08; H05K 1/18

(52) U.S. Cl. CPC A61B 8/12; A61B 8/0891; A61B 8/4254; A61B 8/4494; H05K 1/183; H05K 1/189; A61B 8/445; H05K 2201/10151; H05K 2201/10219

(57) ABSTRACT

A flexible electronic circuit, as may be used in an intravascular catheter, includes a flexible substrate having opposing first and second surfaces, one or more recesses into the first surface, one or more localization elements respectively disposed within the recesses, and a conductive connector pad on the substrate. Additional components, such as thermistors and ultrasound transducers, may also be mounted to the substrate.

### CROSS-REFERENCE TO RELATED APPLICATIONS

This application claims the benefit of U.S. provisional application No. 63/157,232, filed 5 Mar. 2021, which is hereby incorporated by reference as though fully set forth herein.

### BACKGROUND

The present disclosure relates generally to catheters that are used in the human body. In particular, the present disclosure relates to a small form-factor flexible electronic circuit suitable for incorporation into an intravascular catheter, such as within the tip of such a catheter.

Catheters are used for an ever-growing number of procedures. For example, catheters are used for diagnostic, therapeutic, and ablative procedures, to name just a few examples. Typically, the catheter is manipulated through the patient's vasculature and to the intended site, for example, a site within the patient's heart.

It is known to incorporate various electronic components into the tips of intravascular catheters. For instance, piezoelectric ultrasound transducers and supported electronics packages (implemented, for example, as an application-specific integrated circuit (ASIC) or field-programmable gate array (FPGA)) may be mounted within the tip of an intracardiac echocardiography (ICE) catheter, such as disclosed in United States patent application publication no. 2018/0289356 and U.S. provisional application No. 62/987,574, both of which are hereby incorporated by reference as though fully set forth herein. The relatively small size of such components, however, increases the complexity, and therefore the cost, of manufacture.

### BRIEF SUMMARY

Disclosed herein is a flexible electronic circuit, including: a flexible substrate having a first surface and a second surface opposite the first surface, wherein the first surface includes a recess; a localization element disposed within the recess; and a conductive connector pad disposed on the flexible substrate.

The flexible substrate can include a first portion having a first thickness and a second portion having a second thickness thinner than the first thickness. The recess may be positioned within the first portion of the flexible substrate, and the conductive connector pad may be disposed on the second portion of the flexible substrate.

The localization element may include a magnetic localization element and/or an impedance-based localization element.

In embodiments of the disclosure, there are a plurality of recesses and a corresponding plurality of localization elements respectively disposed within the plurality of recesses.

Aspects of the disclosure also include a thermistor mounted to the substrate, for example, within the recess.

Further aspects of the disclosure include an ultrasound transducer assembly mounted to the substrate, for example, on the second surface of the substrate.

Still further aspects of the disclosure include an adhesive overlaying at least a portion of the substrate.

Also disclosed herein is a tip assembly for an intravascular catheter. The tip assembly includes a shell; and a flexible electronic circuit disposed within the shell. In turn, the flexible electronic circuit includes: a flexible substrate having a first surface and a second surface opposite the first surface, wherein the first surface includes a recess; a localization element disposed within the recess; and a conductive connector pad disposed on the flexible substrate.

In embodiments of the tip assembly, the flexible electronic circuit includes a plurality of recesses and a corresponding plurality of localization elements respectively disposed within the plurality of recesses. The flexible electronic circuit may also include a thermistor and/or an ultrasound transducer assembly mounted to the flexible substrate. The thermistor may be mounted in a recess.

The instant disclosure also relates to an intravascular catheter, including an elongate catheter body terminating at a distal end; a tip assembly secured to the distal end of the elongate catheter body; and a flexible electronic circuit disposed within the tip assembly. The flexible electronic circuit includes: a flexible substrate having a first surface and a second surface opposite the first surface, wherein the first surface includes a recess; a localization element disposed within the recess; and a conductive connector pad disposed on the flexible substrate.

In embodiments of the intravascular catheter, the flexible electronic circuit includes a plurality of recesses and a corresponding plurality of localization elements respectively disposed within the plurality of recesses. The flexible electronic circuit may also include a thermistor and/or an ultrasound transducer assembly mounted to the flexible substrate. The thermistor may be mounted in a recess.

The foregoing and other aspects, features, details, utilities, and advantages of the present invention will be apparent from reading the following description and claims, and from reviewing the accompanying drawings.

### BRIEF DESCRIPTION OF THE DRAWINGS

FIG. 1 is a perspective view of a representative catheter according to aspects of the instant disclosure.

FIG. 2 schematically illustrates the distal region of the representative catheter of FIG. 1, including the interior of the tip portion.

FIG. 3A is a top view of a flexible electronic circuit according to aspects of the instant disclosure.

FIG. 3B is a side view of a flexible electronic circuit according to aspects of the instant disclosure.

FIG. 4 illustrates a flexible electronic circuit assembly according to embodiments disclosed herein.

While multiple embodiments are disclosed, still other embodiments of the present disclosure will become apparent to those skilled in the art from the following detailed description, which shows and describes illustrative embodiments. Accordingly, the drawings and detailed description are to be regarded as illustrative in nature and not restrictive.

### DETAILED DESCRIPTION

Aspects of the instant disclosure relate to flexible electronic circuits suitable for use to connect electronics packages integrated into the distal ends of intravascular catheters (e.g., positioned within the distal tip assemblies of such catheters). Those of ordinary skill in the art will appreciate that the teachings herein can be applied to good advantage in connection with various types of catheters, including, but not limited to, intracardiac echocardiography (ICE) catheters.

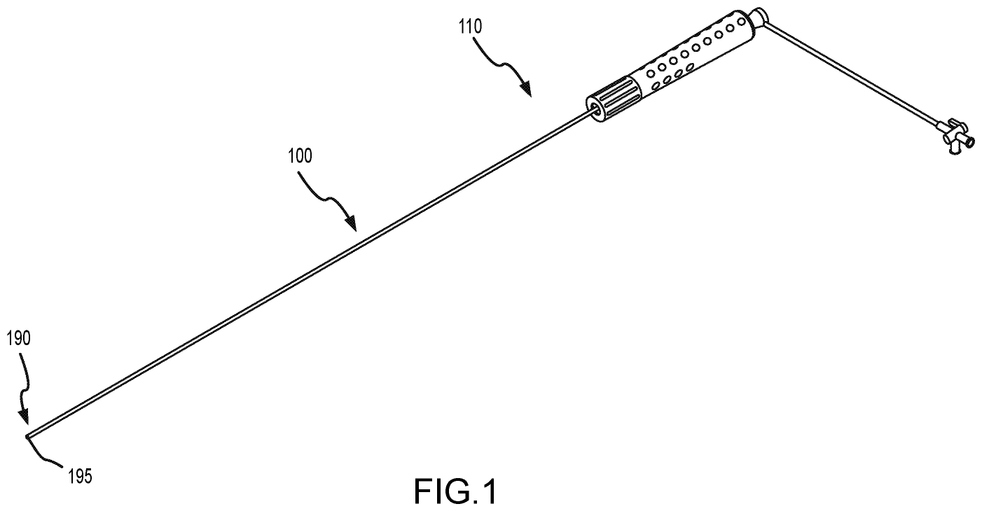

For purposes of illustration, FIG. 1 depicts a perspective view of a representative catheter 100, including a shaft 105 having a proximal portion 110 and a distal portion 190, which terminates in a tip 195. Insofar as the basic construction of catheter 100 will be familiar to those of ordinary skill in the art, the details thereof will be omitted herein, except to the extent relevant to an understanding of the instant disclosure.

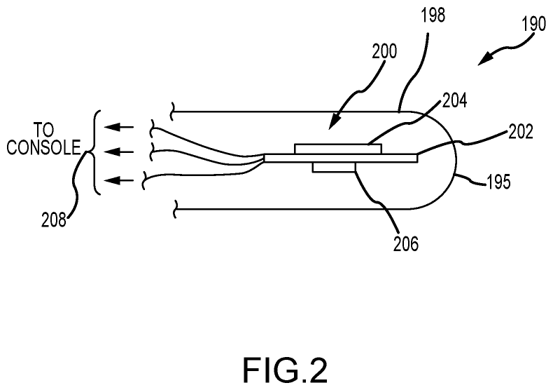

FIG. 2 is a schematic representation of the interior of distal portion 190 of catheter 100, including tip 195. Tip 195 can include a shell 198 and a flexible electronic circuit 200 disposed within shell 198.

Flexible electronic circuit 200, in turn, includes a flexible substrate 202 and a transducer package (e.g., electronics package 204 and ultrasound transducer assembly 206) mounted to substrate 202. A wiring harness 208, including a plurality of wires, interconnects flexible electronic circuit 200 to an external device, such as an electroanatomical mapping system (e.g., the EnSite Precision™ Cardiac Mapping System of Abbott Laboratories) or a catheter interface module. Only three wires are shown within wiring harness 208, but it should be understood that this is merely exemplary and that more or fewer wires may be used in a device according to the present teachings.

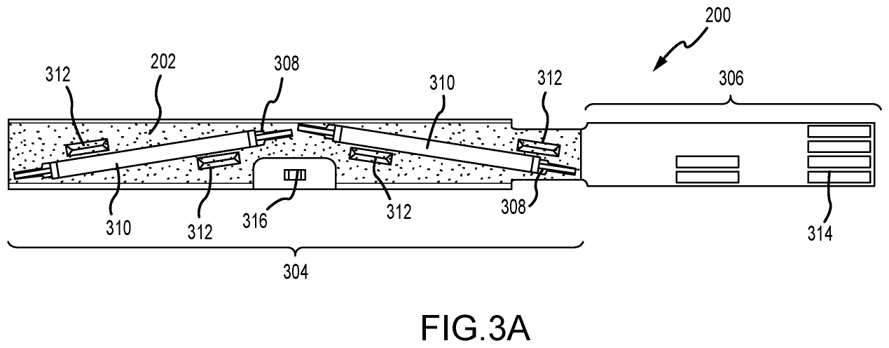

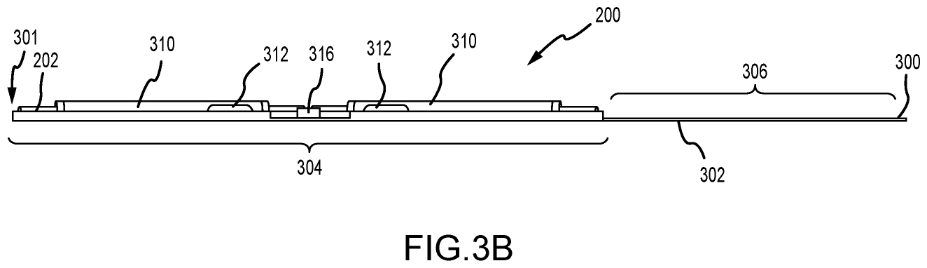

Various details of flexible electronic circuit 200 are shown in FIGS. 3A and 3B. In general, substrate 202 includes a first surface (visible in the plane of FIG. 3A) and a second surface opposite the first surface. The thickness of substrate 202 at any given point is defined between the first and second surfaces at that point.

More particularly, and as best illustrated in FIG. 3B, the thickness of substrate 202 can vary along its length. For instance, in embodiments of the disclosure, substrate 202 includes a thicker first portion 304 (e.g., to support localization elements) and a thinner second portion 306 (e.g., to increase flexibility at the point of interconnection to wiring harness 208). First and second surfaces 301, 302, respectively, define the thickness of the thicker first portion 304 of substrate 202, while first and second surfaces 300, 302, respectively, define the thickness of the thinner second portion 306 of substrate 202. It is contemplated that the thicker first portion 304 of substrate 202 may be formed by bonding one or more additional flexible circuit layers to first surface 300 to build up the additional thickness.

First surface 301 of substrate 202 also includes a recess 308 (e.g., a trench or groove) into the thickness of substrate 202. Typically, although not necessarily, recess 308 will be located within the thicker first portion 304 of substrate 202.

Recess 308 facilitates reliable and accurate positioning of components on substrate 202. In embodiments of the disclosure, a pair of localization elements 310 (e.g., magnetic or impedance-based localization elements) are mounted to substrate 202 as part of electronics package 204 in a respective pair of recesses 308. For example, a pair of five degree of freedom (5DOF) magnetic localization elements 310, mounted at a slight angle with respect to each other within corresponding recesses 308, as shown in FIG. 3A, creates a six degree of freedom (6DOF) localization sensor, as described in United States patent application publication no. 2018/0289356. Solder pads 312 provide electrical connection to localization elements 310.

One or more conductive connector pads 314 are disposed on substrate 202. Although FIG. 3A depicts conductive connector pads 314 only on first surface 300 of substrate 202, this is merely exemplary and the ordinarily-skilled artisan will appreciate that conductive connector pads 314 may be placed elsewhere on substrate 202 without departing from the scope of the instant disclosure.

Conductive connector pads 314 provide points for interconnection with wiring harness 208. Conductive connectors pads 314 are also conductively coupled to localization elements 310 (e.g., to solder pads 312), so as to carry signals, such as power and communications signals, to and from localization elements 310 and, as discussed in further detail below, a temperature sensing element. Insofar as conductive coupling between the attachment point for wiring harness 208 and flexible circuit components will be familiar to those of ordinary skill in the art, it need not be further described herein.

In embodiments of the disclosure, flexible electronic circuit 200 may include additional components. For example, FIGS. 3A and 3B depict a thermistor 316 mounted to substrate 202. According to aspects of the disclosure, thermistor 316 may be mounted within an additional recess analogous to recesses 308. This not only facilitates reliable and accurate positioning of thermistor 316, but also allows for a quicker thermal response by virtue of its proximity to ultrasound transducer assembly 206 through the correspondingly diminished thickness of substrate 202. Other components, such as power supplies, pre-amplifiers, multiplexors, imaging element drivers, additional localization elements, and the like, may also be mounted to substrate 202.

According to aspects of the disclosure, an adhesive may be applied overlaying at least a portion of first and/or second surfaces 300, 302 of substrate 202.

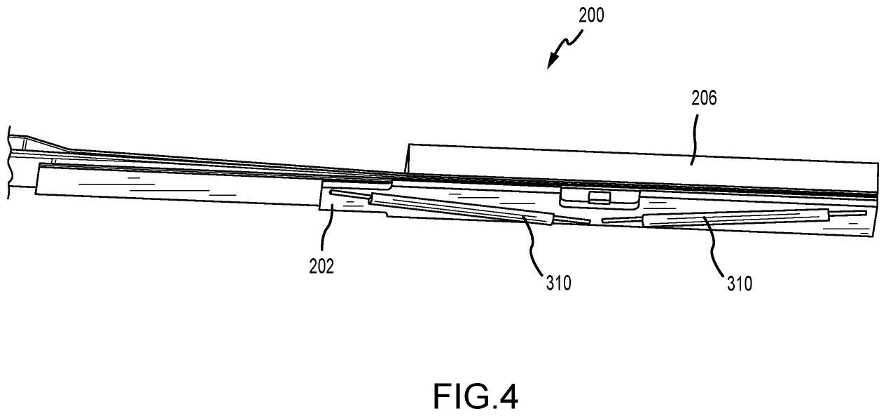

FIG. 4 illustrates flexible electronic circuit 200, with localization elements 310 and ultrasound transducer assembly 206 mounted thereon for assembly into tip 195.

Although several embodiments have been described above with a certain degree of particularity, those skilled in the art could make numerous alterations to the disclosed embodiments without departing from the spirit or scope of this invention.

For example, rather than using individual wires, wiring harness 208 could utilize one or more long flex and/or printed circuits.

All directional references (e.g., upper, lower, upward, downward, left, right, leftward, rightward, top, bottom, above, below, vertical, horizontal, clockwise, and counterclockwise) are only used for identification purposes to aid the reader's understanding of the present invention, and do not create limitations, particularly as to the position, orientation, or use of the invention. Joinder references (e.g., attached, coupled, connected, and the like) are to be construed broadly and may include intermediate members between a connection of elements and relative movement between elements. As such, joinder references do not necessarily infer that two elements are directly connected and in fixed relation to each other.

It is intended that all matter contained in the above description or shown in the accompanying drawings shall be interpreted as illustrative only and not limiting. Changes in detail or structure may be made without departing from the spirit of the invention as defined in the appended claims.

What is claimed is:

1. A flexible electronic circuit, comprising:
a flexible substrate having a first surface and a second surface opposite the first surface, wherein the first surface includes a recess;
a localization element disposed within the recess; and
a conductive connector pad disposed on the flexible substrate.

2. The flexible electronic circuit according to claim 1, wherein the flexible substrate comprises a first portion having a first thickness and a second portion having a second thickness thinner than the first thickness.

3. The flexible electronic circuit according to claim 2, wherein the recess is positioned within the first portion of the flexible substrate.

4. The flexible electronic circuit according to claim 2, wherein the conductive connector pad is disposed on the second portion of the flexible substrate.

5. The flexible electronic circuit according to claim 1, wherein the localization element comprises a magnetic localization element.

6. The flexible electronic circuit according to claim 1, wherein the localization element comprises an impedance-based localization element.

7. The flexible electronic circuit according to claim 1, wherein:
the recess comprises a plurality of recesses; and
the localization element disposed within the recess comprises a plurality of localization elements respectively disposed within the plurality of recesses.

8. The flexible electronic circuit according to claim 1, further comprising a thermistor mounted to the substrate.

9. The flexible electronic circuit according to claim 8, wherein the thermistor is mounted to the flexible substrate within the recess.

10. The flexible electronic circuit according to claim 1, further comprising an ultrasound transducer assembly mounted to the substrate.

11. The flexible electronic circuit according to claim 10, wherein the transducer assembly is mounted to the second surface of the substrate.

12. The flexible electronic circuit according to claim 1, further comprising an adhesive overlaying at least a portion of the substrate.

13. A tip assembly for an intravascular catheter, comprising:
a shell; and
a flexible electronic circuit disposed within the shell, the flexible electronic circuit comprising:
a flexible substrate having a first surface and a second surface opposite the first surface, wherein the first surface includes a recess;
a localization element disposed within the recess; and
a conductive connector pad disposed on the flexible substrate.

14. The tip assembly according to claim 13, wherein:
the recess comprises a plurality of recesses; and
the localization element disposed within the recess comprises a plurality of localization elements respectively disposed within the plurality of recesses.

15. The tip assembly according to claim 13, further comprising a thermistor mounted to the flexible substrate within the recess.

16. The tip assembly according to claim 13, further comprising an ultrasound transducer assembly mounted to the second surface of the flexible substrate.

17. An intravascular catheter, comprising:
an elongate catheter body terminating at a distal end;
a tip assembly secured to the distal end of the elongate catheter body; and
a flexible electronic circuit disposed within the tip assembly, the flexible electronic circuit comprising:
a flexible substrate having a first surface and a second surface opposite the first surface, wherein the first surface includes a recess;
a localization element disposed within the recess; and
a conductive connector pad disposed on the flexible substrate.

18. The intravascular catheter according to claim 17, wherein:
the recess comprises a plurality of recesses; and
the localization element disposed within the recess comprises a plurality of localization elements respectively disposed within the plurality of recesses.

19. The intravascular catheter according to claim 17, further comprising a thermistor mounted to the flexible substrate within the recess.

20. The intravascular catheter according to claim 17, further comprising an ultrasound transducer assembly mounted to the second surface of the flexible substrate.
